(19) United States
(12) Patent Application Publication
(10) Pub. No.: US 2021/0253560 A1
(43) Pub. Date: Aug. 19, 2021

(54) PLANT GROWTH REGULATING COMPOUNDS

(71) Applicant: SYNGENTA PARTICIPATIONS AG, Basel (CH)

(72) Inventors: Alexandre Franco Jean Camille LUMBROSO, Stein (CH); Alain DE MESMAEKER, Stein (CH); Claudio SCREPANTI, Stein (CH)

(73) Assignee: Syngenta Participations AG, Basel (CH)

(21) Appl. No.: 16/981,510

(22) PCT Filed: Mar. 7, 2019
(86) PCT No.: PCT/EP2019/055754

§ 371 Date: Sep. 16, 2020

(30) Foreign Application Priority Data
Mar. 16, 2018 (GB) 1804249.9

(51) Int. Cl. C07D 405/12; C07D 417/14; A01N 43/78; A01N 43/38

(52) U.S. Cl. CPC C07D 405/12; A01N 43/38; A01N 43/78; C07D 417/14

(57) ABSTRACT

The present invention relates to novel strigolactam derivatives, to crop enhancement, plant growth regulator or seed germination promoting compositions comprising these derivatives and to methods of using these derivatives in controlling the growth and physiology of plants and/or promoting the germination of seeds.

### CROSS-REFERENCE TO RELATED APPLICATIONS

This application is a 371 National Stage application of International Application No. PCT/EP2019/055754 filed Mar. 7, 2019 which claims priority to GB 1804249.9, filed Mar. 16, 2018, the entire contents of these applications are hereby incorporated by reference.

The present invention relates to novel strigolactam derivatives, to processes for preparing these derivatives including intermediate compounds, to crop enhancement, plant growth regulator or seed germination promoting compositions comprising these derivatives and to methods of using these derivatives in controlling the growth and physiology of plants and/or promoting the germination of seeds.

Strigolactone derivatives are phytohormones which may have plant growth regulation and seed germination properties. They have previously been described in the literature. Certain known strigolactam derivatives (e.g. see WO2012/080115 and WO2016/193290), may have properties analogous to strigolactones, e.g. plant growth regulation and/or seed germination promotion. For such compounds to be used, in particular, for foliar applications or in seed treatment (e.g. as seed coating components), their binding affinities with the strigolactone receptor D14 are important.

The present invention relates to novel strigolactam derivatives that have improved properties. Benefits of the compounds of the present invention include improved tolerance to abiotic stress, improved seed germination, better regulation of crop growth, improved crop yield, and/or improved physical properties such as chemical, hydrolytic, physical and/or soil stability.

According to the present invention, there is provided a compound of Formula (I):

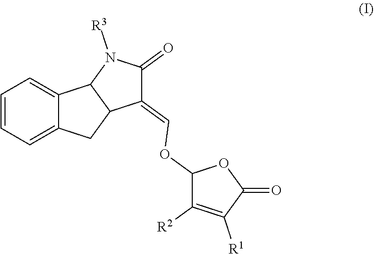

wherein
R¹ and R² are each independently methyl or ethyl; and
R³ is selected from the group consisting of formyl, C₁-C₄ alkylcarbonyl, C₁-C₄ alkoxycarbonyl, C₃-C₈ cycloalkylcarbonyl, C₁-C₄ haloalkylcarbonyl, aryl, heteroaryl, and acetonitrile;
or salts thereof.

The compounds of Formula (I) have been shown to possess better affinity with maize strigolactone receptor (014) as well as improved ability to induce leaf senescence compared to known strigolactam derivatives.

The compounds of Formula (I) may exist in different geometric or optical isomers (diastereoisomers and enantiomers) or tautomeric forms. This invention covers all such isomers and tautomers and mixtures thereof, in all proportions, as well as isotopic forms, such as deuterated compounds. The invention also covers all salts, and metalloidic complexes of the compounds of Formula (I).

Each alkyl moiety either alone or as part of a larger group (such as alkoxycarbonyl, alkylcarbonyl, halogenoalkyl) is a straight or branched chain and is, for example, methyl, ethyl, n-propyl, n-butyl, iso-propyl, n-butyl, sec-butyl, iso-butyl, tert-butyl.

Unless otherwise indicated, cycloalkyl may be mono- or bi-cyclic, may be optionally substituted by one or more C₁-C₄ alkyl groups, and contain 3 to 8 carbon atoms. Examples of cycloalkyl include cyclopropyl, 1-methylcyclopropyl, 2-methylcyclopropyl, cyclobutyl, cyclopentyl and cyclohexyl.

The term “haloalkyl” (either alone or as part of a larger group, such as haloalkoxy or haloalkylthio), as used herein, are alkyl groups which are substituted with one or more of the same or different halogen atoms and are, for example, —CF₃, —CF₂C₁, —CH₂CF₃ or —CH₂CHF₂.

Halogen is fluorine (F), chlorine (Cl), bromine (Br) or iodine (I).

The term “aryl”, as used herein, refers to a ring system which may be mono, bi or tricyclic. Examples of such rings include phenyl, naphthalenyl, anthracenyl, indenyl or phenanthrenyl.

The term “heteroaryl”, as used herein, refers to an aromatic ring system containing from one to four heteroatoms selected from N, O, and S, wherein the nitrogen and sulfur atoms are optionally oxidized, for example having 5, 6, 9 or 10 members, and consisting either of a single ring or of two or more fused rings. Single rings may contain up to three heteroatoms, and bicyclic systems up to four heteroatoms, which will preferably be chosen from nitrogen, oxygen and sulfur. Examples of such groups include pyridyl, pyridazinyl, pyrimidinyl, pyrazinyl, furanyl, thienyl, oxazolyl, isoxazolyl, oxadiazolyl, thiazolyl, isothiazolyl, thiadiazolyl, pyrrolyl, pyrazolyl, imidazolyl, triazolyl and tetrazolyl.

In one embodiment, R³ is selected from the group consisting of formyl, C₁-C₄ alkylcarbonyl, C₁-C₄ alkoxycarbonyl, C₃-C₈ cycloalkylcarbonyl, C₁-C₄ haloalkylcarbonyl, aryl, heteroaryl, and acetonitrile.

In one embodiment, R³ is selected from the group consisting of formyl, C₃-C₈ cycloalkylcarbonyl, C₁-C₄ haloalkylcarbonyl, and acetonitrile.

In one embodiment, R³ is selected from the group consisting of phenyl, C₁-C₄ alkylcarbonyl, heteroaryl, and acetonitrile.

In one embodiment, R³ is selected from the group consisting of formyl, acetyl, phenyl, 2-thiazolyl, and acetonitrile.

In one embodiment, R¹ and R² are both methyl.

In one embodiment, R³ is C₁-C₄ alkyl(CO)—.

In one embodiment, R³ is C₁-C₄ haloalkyl(CO)—.

In one embodiment, R³ is formyl.

In one embodiment, R³ is phenyl.

In one embodiment, R³ is 2-thiazolyl.

In one embodiment, R³ is acetonitrile.

In one embodiment, R³ is acetyl.

Preferably, the compound of Formula (I) has the structure of Formula (IA-1):

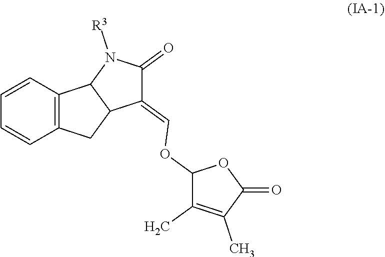

Table 1 below includes examples IA-1 to IA-20 of compounds of Formula (I) according to the invention:

TABLE 1
(I)

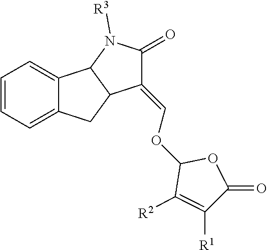

| Compound | R¹ | R² | R³ |
|---|---|---|---|
| IA-1 | —CH₃ | —CH₃ | CH₃(CO)— |
| IA-2 | —CH₃ | —CH₃ | CH₃CH₂(CO)— |
| IA-3 | —CH₃ | —CH₃ | CH₃(CH₂)₂(CO)— |
| IA-4 | —CH₃ | —CH₃ | CF₃(CO)— |
| IA-5 | —CH₃ | —CH₃ | CF₃CH₂(CO)— |
| IA-6 | —CH₃ | —CH₃ | cC₃H₅(CO)— |
| IA-7 | —CH₃ | —CH₃ | 2-Thiazolyl |
| IA-8 | —CH₃ | —CH₃ | Phenyl |
| IA-9 | —CH₃ | —CH₃ | 3,5-(CF₃)₂Ph |
| IA-10 | —CH₃ | —CH₃ | —CH₂CN |
| IA-11 | —C₂H₅ | —C₂H₅ | CH₃(CO)— |
| IA-12 | —C₂H₅ | —C₂H₅ | CH₃CH₂(CO)— |
| IA-13 | —C₂H₅ | —C₂H₅ | CH₃(CH₂)₂(CO)— |
| IA-14 | —C₂H₅ | —C₂H₅ | CF₃(CO)— |
| IA-15 | —C₂H₅ | —C₂H₅ | CF₃CH₂(CO)— |
| IA-16 | —C₂H₅ | —C₂H₅ | cC₃H₅(CO)— |
| IA-17 | —C₂H₅ | —C₂H₅ | 2-Thiazolyl |
| IA-18 | —C₂H₅ | —C₂H₅ | Phenyl |
| IA-19 | —C₂H₅ | —C₂H₅ | 3,5-(CF₃)₂Ph |
| IA-20 | —C₂H₅ | —C₂H₅ | —CH₂CN |

In one embodiment, the compounds of the present invention are applied in combination with an agriculturally acceptable adjuvant. In particular, there is provided a composition comprising a compound of the present invention and an agriculturally acceptable adjuvant. There may also be mentioned an agrochemical composition comprising a compound of the present invention.

In one aspect of the invention, there is provided a crop yield enhancing, abiotic stress management, plant growth regulator or seed germination promoting composition, comprising a compound of the present invention, and optionally, an agriculturally acceptable formulation adjuvant.

In one aspect of the invention, there is provided a mixture comprising a compound of the present invention and at least one further active ingredient. The further active ingredient may be, for example an acaricide, bactericide, fungicide, herbicide, insecticide, miticide, molluscicide, nematicide, plant activator, plant growth regulator, biostimulant, rodenticide, safener, synergist, crop enhancing agent or an active ingredient that improves tolerance of plants to abiotic stress conditions.

The present invention provides a method for enhancing the yield of plants, wherein the method comprises applying to the plant, plant part, plant propagation material, or plant growing locus a compound, composition or mixture according to the present invention. In one embodiment, the compound, composition or mixture of the present invention is applied in a yield boosting amount.

The present invention provides a method of improving the tolerance of a plant to abiotic stress factors, wherein the method comprises applying to the plant, plant part, plant propagation material, or plant growing locus a compound, composition or mixture according to the present invention. In one embodiment the abiotic stress is cold, salt, drought and/or osmotic stress. In a further embodiment, the abiotic stress is drought. In one embodiment, the compound, composition or mixture of the present invention is applied in an amount that improves tolerance to abiotic stress factors.

The present invention provides a method for regulating or improving the growth of a plant, wherein the method comprises applying to the plant, plant part, plant propagation material, or plant growing locus a compound, composition or mixture according to the present invention. In one embodiment, plant growth is regulated or improved when the plant is subject to abiotic stress conditions. In one embodiment, the compound, composition or mixture of the present invention is applied in a plant growth regulating amount.

The present invention also provides a method for promoting seed germination or emergence of a plant, comprising applying to the seed, or a locus containing seeds, a compound, composition or mixture according to the present invention. Germination or emergence are stimulated, for example through faster or more uniform germination or emergence. In one embodiment, the compound, composition or mixture of the present invention is applied in a seed germination promoting amount.

The present invention also provides a method for controlling weeds, comprising applying to a locus containing weed seeds, a seed germination promoting amount of a composition according to the second aspect of the invention, allowing the seeds to germinate, and then applying to the locus a post-emergence herbicide.

In a further aspect of the invention, there is provided the use of a compound of Formula (I) according to the invention as a crop yield enhancer, plant growth regulator or a seed germination promoter.

The present invention also provides a method for safening a plant against phytotoxic effects of chemicals, comprising applying to the plant, plant part, plant propagation material, or plant growing locus a compound, composition or mixture according to the present invention.

The present invention also provides a method for accelerating senescence of plant leaves, comprising applying to the plant, plant part, plant propagation material, or plant growing locus a compound, composition or mixture according to the present invention. In one embodiment, the compound, composition or mixture of the present invention is applied in a leaf senescence regulating amount.

Suitably the compound or composition is applied in an amount sufficient to elicit the desired response.

In a further aspect of the invention, there is provided a method of treating a plant propagation material comprising applying to the plant propagation material a composition according to the invention in an amount effective to promote germination, to enhance the yield and/or regulate plant growth.

In a further aspect of the invention, there is provided a plant propagation material treated with a compound of Formula (I) according to the invention, or a composition according to the invention.

The present invention may also provide method to improve nutrient (such as nitrogen or sugar) recycling and remobilization in plants via leaf senescence.

According to the present invention, “regulating or improving the growth of a plant” means an improvement in plant vigour, an improvement in plant quality, improved tolerance to stress factors, and/or improved input use efficiency.

An ‘improvement in plant vigour’ means that certain traits are improved qualitatively or quantitatively when compared with the same trait in a control plant which has been grown under the same conditions in the absence of the method of the invention. Such traits include, but are not limited to, early and/or improved germination, improved emergence, the ability to use less seeds, increased root growth, a more developed root system, increased root nodulation, increased shoot growth, increased tillering, stronger tillers, more productive tillers, increased or improved plant stand, less plant verse (lodging), an increase and/or improvement in plant height, an increase in plant weight (fresh or dry), bigger leaf blades, greener leaf colour, increased pigment content, increased photosynthetic activity, earlier flowering, longer panicles, early grain maturity, increased seed, fruit or pod size, increased pod or ear number, increased seed number per pod or ear, increased seed mass, enhanced seed filling, less dead basal leaves, delay of senescence, improved vitality of the plant, increased levels of amino acids in storage tissues and/or less inputs needed (e.g. less fertiliser, water and/or labour needed). A plant with improved vigour may have an increase in any of the aforementioned traits or any combination or two or more of the aforementioned traits.

An ‘improvement in plant quality’ means that certain traits are improved qualitatively or quantitatively when compared with the same trait in a control plant which has been grown under the same conditions in the absence of the method of the invention. Such traits include, but are not limited to, improved visual appearance of the plant, reduced ethylene (reduced production and/or inhibition of reception), improved quality of harvested material, e.g. seeds, fruits, leaves, vegetables (such improved quality may manifest as improved visual appearance of the harvested material), improved carbohydrate content (e.g. increased quantities of sugar and/or starch, improved sugar acid ratio, reduction of reducing sugars, increased rate of development of sugar), improved protein content, improved oil content and composition, improved nutritional value, reduction in anti-nutritional compounds, improved organoleptic properties (e.g. improved taste) and/or improved consumer health benefits (e.g. increased levels of vitamins and anti-oxidants)), improved post-harvest characteristics (e.g. enhanced shelf-life and/or storage stability, easier processability, easier extraction of compounds), more homogenous crop development (e.g. synchronised germination, flowering and/or fruiting of plants), and/or improved seed quality (e.g. for use in following seasons). A plant with improved quality may have an increase in any of the aforementioned traits or any combination or two or more of the aforementioned traits.

An ‘improved tolerance to stress factors’ means that certain traits are improved qualitatively or quantitatively when compared with the same trait in a control plant which has been grown under the same conditions in the absence of the method of the invention. Such traits include, but are not limited to, an increased tolerance and/or resistance to biotic and/or abiotic stress factors, and in particular abiotic stress factors which cause sub-optimal growing conditions such as drought (e.g. any stress which leads to a lack of water content in plants, a lack of water uptake potential or a reduction in the water supply to plants), cold exposure, heat exposure, osmotic stress, UV stress, flooding, increased salinity (e.g. in the soil), increased mineral exposure, ozone exposure, high light exposure and/or limited availability of nutrients (e.g. nitrogen and/or phosphorus nutrients). A plant with improved tolerance to stress factors may have an increase in any of the aforementioned traits or any combination or two or more of the aforementioned traits. In the case of drought and nutrient stress, such improved tolerances may be due to, for example, more efficient uptake, use or retention of water and nutrients. In particular, the compounds or compositions of the present invention are useful to improve tolerance to drought stress.

An ‘improved input use efficiency’ means that the plants are able to grow more effectively using given levels of inputs compared to the grown of control plants which are grown under the same conditions in the absence of the method of the invention. In particular, the inputs include, but are not limited to fertiliser (such as nitrogen, phosphorous, potassium, micronutrients), light and water. A plant with improved input use efficiency may have an improved use of any of the aforementioned inputs or any combination of two or more of the aforementioned inputs.

Other effects of regulating or improving the growth of a crop include a decrease in plant height, or reduction in tillering, which are beneficial features in crops or conditions where it is desirable to have less biomass and fewer tillers.

Any or all of the above crop enhancements may lead to an improved yield by improving e.g. plant physiology, plant growth and development and/or plant architecture. In the context of the present invention ‘yield’ includes, but is not limited to, (i) an increase in biomass production, grain yield, starch content, oil content and/or protein content, which may result from (a) an increase in the amount produced by the plant per se or (b) an improved ability to harvest plant matter, (ii) an improvement in the composition of the harvested material (e.g. improved sugar acid ratios, improved oil composition, increased nutritional value, reduction of anti-nutritional compounds, increased consumer health benefits) and/or (iii) an increased/facilitated ability to harvest the crop, improved processability of the crop and/or better storage stability/shelf life. Increased yield of an agricultural plant means that, where it is possible to take a quantitative measurement, the yield of a product of the respective plant is increased by a measurable amount over the yield of the same product of the plant produced under the same conditions, but without application of the present invention. According to the present invention, it is preferred that the yield be increased by at least 0.5%, more preferred at least 1%, even more preferred at least 2%, still more preferred at least 4%, preferably 5% or even more.

Any or all of the above crop enhancements may also lead to an improved utilisation of land, i.e. land which was previously unavailable or sub-optimal for cultivation may become available. For example, plants which show an increased ability to survive in drought conditions, may be able to be cultivated in areas of sub-optimal rainfall, e.g. perhaps on the fringe of a desert or even the desert itself.

In one aspect of the present invention, crop enhancements are made in the substantial absence of pressure from pests and/or diseases and/or abiotic stress. In a further aspect of the present invention, improvements in plant vigour, stress tolerance, quality and/or yield are made in the substantial absence of pressure from pests and/or diseases. For example pests and/or diseases may be controlled by a pesticidal treatment that is applied prior to, or at the same time as, the method of the present invention. In a still further aspect of the present invention, improvements in plant vigour, stress tolerance, quality and/or yield are made in the absence of pest and/or disease pressure. In a further embodiment, improvements in plant vigour, quality and/or yield are made in the absence, or substantial absence, of abiotic stress.

The present invention also provides the use of a compound or composition of the present invention for improving the tolerance of a plant to abiotic stress factors, regulating or improving the growth of a plant, promoting seed germination and/or safening a plant against phytotoxic effects of chemicals.

The present invention also provides the use of a compound, composition or mixture of the present invention, for stimulating seed germination and/or seedling emergence, for example through faster or more uniform germination or emergence.

The present invention provides the use of a compound, composition or mixture of the present invention, for improving the tolerance of a plant to abiotic stress factors. In one embodiment the abiotic stress is cold, salt, drought and/or osmotic stress.

Preferably, the crop yield enhancing, plant growth regulator or seed germination promoting composition according to the invention is a composition that is a seed treatment composition or a seed coating composition. The compositions according to the invention may also further comprise an insecticidal, acaracidal, nematicidal or fungicidal active ingredient.

Preferably, the compound of Formula (I) according to the invention is for use in a foliar or a seed treatment composition.

Preferably, the plant propagation material of the invention is a seed. In one embodiment the seed is a corn (maize) seed.

The compound of Formula (I) according to the invention can be used as a crop/yield enhancer, a plant growth regulator or seed germination promoter by itself, but is generally formulated into a crop/yield enhancement, plant growth regulation or seed germination promotion composition using formulation adjuvants, such as carriers, solvents and surface-active agents (SFAs). The composition can be in the form of concentrates which are diluted prior to use, although ready-to-use compositions can also be utilised. The final dilution is usually made with water, but can be made instead of, or in addition to, water, with, for example, liquid fertilisers, other active ingredients (e.g. insecticidal, acaracidal, nematicidal or fungicidal components), micronutrients, biological organisms, oil or solvents.

The compositions generally comprise from 0.1 to 99% by weight, especially from 0.1 to 95% by weight, of a compound of Formula (I) and from 1 to 99.9% by weight of a formulation adjuvant, which preferably includes from 0 to 25% by weight of an SFA.

The compositions can be chosen from a number of formulation types, many of which are known from the Manual on Development and Use of FAO Specifications for Plant Protection Products, 5th Edition, 1999.

These include dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, capsule suspensions (CS) and seed treatment formulations. The formulation type chosen in any instance will depend upon the particular purpose envisaged and the physical, chemical and biological properties of the compound of Formula (I).

Dustable powders (DP) may be prepared by mixing a compound of Formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers) and mechanically grinding the mixture to a fine powder.

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fullers earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether. These solutions may contain a surface active agent (for example to improve water dilution or prevent crystallisation in a spray tank). Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of Formula (I) is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. The compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of the compound of Formula (I). Such additives include SFAs, spray additives based on oils, for example certain mineral oils or natural plant oils (such as soy bean and rape seed oil), and blends of these with other bio-enhancing adjuvants (ingredients which may aid or modify the action of a compound of Formula (I)).

Wetting agents, dispersing agents and emulsifying agents may be SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

In addition, further, other biocidally-active ingredients or compositions may be combined with the compositions of the invention and used in the methods of the invention and applied simultaneously or sequentially with the compositions of the invention. When applied simultaneously, these further active ingredients may be formulated together with the compositions of the invention or mixed in, for example, the spray tank. These further biocidally active ingredients may be fungicides, insecticides, bactericides, acaricides, nematicides and/or other plant growth regulators. Pesticidal agents are referred to herein using their common name are known, for example, from “The Pesticide Manual”, 15th Ed., British Crop Protection Council 2009.

In the methods for regulating the growth of plants in a locus and for promoting the germination of seeds according to the present invention, the application is generally made by spraying the composition, typically by tractor mounted sprayer for large areas, but other methods such as dusting (for powders), drip or drench can also be used. Alternatively, the composition may be applied in furrow or directly to a seed before or at the time of planting. In the method for promoting the germination of seeds according to the present invention, the compound of Formula (I) may be incorporated as a component in a seed treatment composition.

The compound of Formula (I) or composition of the present invention may be applied to a plant, part of the plant, plant organ, plant propagation material or a surrounding area thereof.

In one embodiment, the invention relates to a method of treating a plant propagation material comprising applying to the plant propagation material a composition of the present invention in an amount effective to enhance the yield, promote germination and/or regulate plant growth. The invention also relates to a plant propagation material treated with a compound of Formula (I) or a composition of the present invention. Preferably, the plant propagation material is a seed.

The term “plant propagation material” denotes all the generative parts of the plant, such as seeds, which can be used for the multiplication of the latter and vegetative plant materials such as cuttings and tubers. In particular, there may be mentioned the seeds, roots, fruits, tubers, bulbs, and rhizomes.

The term “plants” refers to all physical parts of a plant, including seeds, seedlings, saplings, roots, tubers, stems, stalks, foliage, and fruits.

The term “locus” as used herein means fields in or on which plants are growing, or where seeds of cultivated plants are sown, or where seed will be placed into the soil. It includes soil, seeds, and seedlings, as well as established vegetation.

Methods for applying active ingredients to plant propagation material, especially seeds, are known in the art, and include dressing, coating, pelleting and soaking application methods of the propagation material. The treatment can be applied to the seed at any time between harvest of the seed and sowing of the seed or during the sowing process. The seed may also be primed either before or after the treatment. The compound of Formula (I) may optionally be applied in combination with a controlled release coating or technology so that the compound is released over time.

The composition of the present invention may be applied pre-emergence or post-emergence. Suitably, where the composition is being used to regulate the growth of crop plants or to enhance the yield, it may be applied pre- or post-emergence, but preferably post-emergence of the crop. Where the composition is used to promote the germination of seeds, it may be applied pre-emergence.

The rates of application of the compound of Formula (I) may vary within wide limits and depend on the nature of the soil, the method of application (pre- or post-emergence; seed dressing; application to the seed furrow; no tillage application, etc.), the crop plant, the prevailing climatic conditions, and other factors governed by the method of application, the time of application and the target crop. For foliar or drench application, the compound of Formula (I) according to the invention is generally applied at a rate of from 1 to 2000 g/ha, especially from 5 to 1000 g/ha. For seed treatment, the rate of application is generally between 0.0005 and 150 g per 100 kg of seed.

Plants in which the composition according to the invention can be used include crops such as cereals (for example wheat, barley, rye, oats); beet (for example sugar beet or fodder beet); fruits (for example pomes, stone fruits or soft fruits, such as apples, pears, plums, peaches, almonds, cherries, strawberries, raspberries or blackberries); leguminous plants (for example beans, lentils, peas or soybeans); oil plants (for example rape, mustard, poppy, olives, sunflowers, coconut, castor oil plants, cocoa beans or groundnuts); cucumber plants (for example marrows, cucumbers or melons); fibre plants (for example cotton, flax, hemp or jute); citrus fruit (for example oranges, lemons, grapefruit or mandarins); vegetables (for example spinach, lettuce, asparagus, cabbages, carrots, onions, tomatoes, potatoes, cucurbits or paprika); lauraceae (for example avocados, cinnamon or camphor); maize; rice; tobacco; nuts; coffee; sugar cane; tea; vines; hops; durian; bananas; natural rubber plants; turf or ornamentals (for example flowers, shrubs, broad-leaved trees or evergreens such as conifers). This list does not represent any limitation.

The invention may also be used to regulate the growth, or promote the germination of seeds of non-crop plants, for example to facilitate weed control by synchronizing germination.

Crops are to be understood as also including those crops which have been modified by conventional methods of breeding or by genetic engineering. For example, the invention may be used in conjunction with crops that have been rendered tolerant to herbicides or classes of herbicides (e.g. ALS-, GS-, EPSPS-, PPO-, ACCase- and HPPD-inhibitors). An example of a crop that has been rendered tolerant to imidazolinones, e.g., imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®. Methods of rendering crop plants tolerant to HPPD-inhibitors are known; for example the crop plant is transgenic in respect of a polynucleotide comprising a DNA sequence which encodes an HPPD-inhibitor resistant HPPD enzyme derived from a bacterium, more particularly from Pseudomonas fluorescens or Shewanella colwelliana, or from a plant, more particularly, derived from a monocot plant or, yet more particularly, from a barley, maize, wheat, rice, Brachiaria, Chenchrus, Lolium, Festuca, Setaria, Eleusine, Sorghum or Avena species.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood to include those which are obtained by conventional methods of breeding or genetic engineering and contain so-called output traits (e.g., improved storage stability, higher nutritional value and improved flavour).

### EXAMPLES

The Examples which follow serve to illustrate the invention.

### Compound Synthesis and Characterisation

The following abbreviations are used throughout this section: s=singlet; bs=broad singlet; d=doublet; dd=double doublet; dt=double triplet; bd=broad doublet; t=triplet; dt=double triplet; bt=broad triplet; tt=triple triplet; q=quartet; m=multiplet; Me=methyl; DME=Dimethoxyethane; RT=retention time, MH⁺=molecular cation (i.e. measured molecular weight).

The following HPLC-MS method was used for the analysis of the compounds: Spectra were recorded on a ZQ Mass Spectrometer from Waters (Single quadrupole mass spectrometer) equipped with an electrospray source (Polarity: positive or negative ions, Capillary: 3.00 kV, Cone: 30.00 V, Extractor: 2.00 V, Source Temperature: 100° C., Desolvation Temperature: 250° C., Cone Gas Flow: 50 L/Hr, Desolvation Gas Flow: 400 L/Hr, Mass range: 100 to 900 Da and an Acquity UPLC from Waters (Solvent degasser, binary pump, heated column compartment and diode-array detector. Column: Waters UPLC HSS T3, 1.8 μm, 30×2.1 mm, Temp: 60° C., flow rate 0.85 mL/min; DAD Wavelength range (nm): 210 to 500) Solvent Gradient: A=H₂O+5% MeOH+0.05% HCOOH, B=Acetonitrile+0.05% HCOOH) gradient: 0 min 10% B; 0-1.2 min 100% B; 1.2-1.50 min 100% B.

Compounds of the invention were prepared in accordance with Preparation Examples 1 and 2.

### Preparation Example 1: (3E)-1-acetyl-3-[(3,4-dimethyl-5-oxo-2H-furan-2-yl)oxymethylene]-4,8b-dihydro-3aH-indeno[1,2-b]pyrrol-2-one (IA-1)

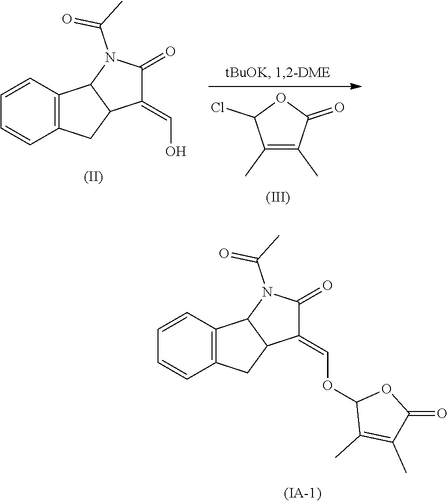

Known compound of formula (II) (WO2012/080115) (4.5 g, 18 mmol) was dissolved in 1,2-DME (140 mL), cooled to 0° C. and tBuOK was added (2.5 g, 22 mmol, 1.2 eq). After 35 minutes, known compound of formula (III) (WO2016/193290) was added dropwise. After 20 minutes at 0° C., the reaction mixture was slowly warm to room temp and stirred for additional 5 hours. The reaction mixture, was poured into a saturated aqueous NH₄Cl solution and diluted with ethyl acetate. The phases were separated and the organic layer was dried over sodium sulfate and concentrated under vacuum. The resulting crude oil was purified by flash chromatography on SiO₂ affording compound of formula (IA-1) as a white solid and mixture of diastereoisomers (2.5 g, 7.1 mmol, 38% yield). LCMS (Method A): RT 0.99 min; ES⁺ 354 (M+H⁺); ¹H NMR (400 MHz, CDCl₃) (for both diastereoisomers) δ 1.93 (m, 6H), 2.04 (m, 3H), 2.07 (m, 3H), 2.57 (s, 6H), 3.19 (m, 2H), 3.32-3.43 (m, 2H), 3.76 (m, 2H), 5.91 (m, 1H), 5.93 (m, 1H), 5.97 (m, 2H), 7.16-7.23 (m, 4H), 7.24-7.30 (m, 2H), 7.44 (dd, 2H), 7.62-7.68 (m, 2H).

### Compounds IA-7, IA-8 and IA-10 were Prepared Using Similar Procedure from Known Intermediates II-7, II-8 and II-10 Described in WO2012/080115 (Rt=Retention Time)

| Cpd No. | Structure | Name | LCMS |
|---|---|---|---|
| IA-7 | 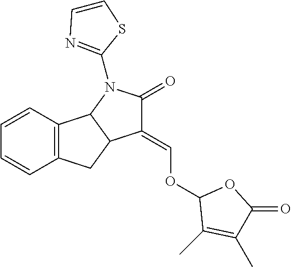 [SMILES: CC1=C(C)C(O/C=C2/C(=O)N(c3nccs3)C3c4ccccc4CC23)OC1=O] | (3E)-3-[(3,4-dimethyl-5-oxo- 2H-furan-2-yl)oxymethylene]- 1-thiazol-2-yl-4,8b-dihydro- 3aH-indeno[1,2-b]pyrrol-2- one | Rₜ = 1.71 min (Method A); ES⁺ 395 (M + H⁺) |
| IA-8 | 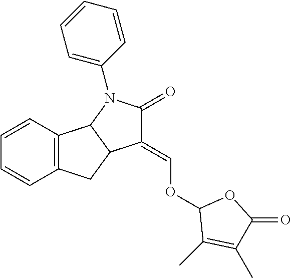 [SMILES: CC1=C(C)C(O/C=C2/C(=O)N(c3ccccc3)C3c4ccccc4CC23)OC1=O] | (3E)-3-[(3,4-dimethyl-5-oxo- 2H-furan-2-yl)oxymethylene]- 1-phenyl-4,8b-dihydro-3aH- indeno[1,2-b]pyrrol-2-one | Rₜ = 1.05 min (Method A); ES⁺ 388 (M + H⁺) |
| IA-10-E | 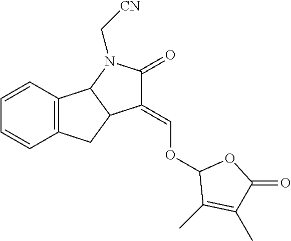 [SMILES: CC1=C(C)C(O/C=C2/C(=O)N(CC#N)C3c4ccccc4CC23)OC1=O] | 2-[(3E)-3-[(3,4-dimethyl-5- oxo-2H-furan-2- yl)oxymethylene]-2-oxo-4,8b- dihydro-3aH-indeno[1,2- b[pyrrol-1-yl]acetonitrile | Rₜ = 0.91 min (Method A); ES⁺ 351 (M + H⁺) |
| IA-10-Z | 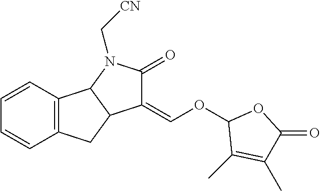 [SMILES: CC1=C(C)C(O/C=C2\C(=O)N(CC#N)C3c4ccccc4CC23)OC1=O] | 2-[(3Z)-3-[(3,4-dimethyl-5- oxo-2H-furan-2- yl)oxymethylene]-2-oxo-4,8b- dihydro-3aH-indeno[1,2- b]pyrrol-1-yl]acetonitrile | Rₜ = 0.88/0.89 min (Method A); ES⁺ 351 (M + H⁺) |

### Preparation Example 2: (3E)-3-[(3,4-dimethyl-5-oxo-2H-furan-2-yl)oxymethylene]-1-propanoyl-4,8b-dihydro-3aH-indeno[1,2-b]pyrrol-2-one (IA-2)

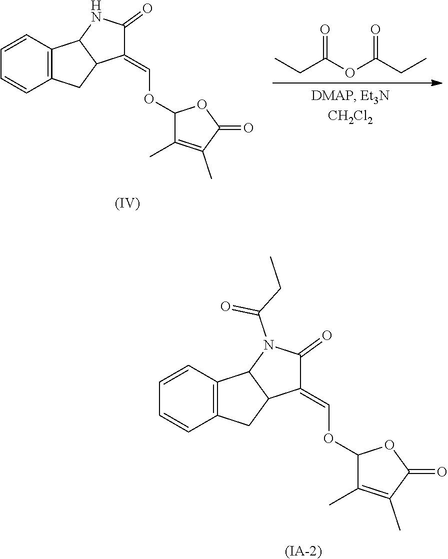

To a degassed solution of known compound of formula (IV) (0.2 g, 0.64 mmol) in dichloromethane (5.8 mL) was added dimethylamino pyridine (DMAP) (0.004 g, 0.003 mmol) and Et₃N (0.36 mL, 2.57 mmol) followed by dropwise addition of propanoyl propanoate (0.1 g, 0.77 mmol) at r.t. The reaction mixture was then stirred overnight at reflux, poured into sat agNH₄Cl solution (after cooling to room temperature) and diluted with ethyl acetate. The phases were separated and the organic layer was dried over sodium sulfate and concentrated under vacuum. The crude reaction mixture was purified by flash chromatography affording compound (IA-2) in 74% yield (0.17 g, 0.48 mmol). LCMS (Method A): RT 1.06 min; ES⁺368 (M+H⁺)

### Compounds I-3, IA-5 and IA-6 were Prepared Via a Similar Method Using the Appropriate Anhydride or Acyl Chloride (Rt=Retention Time)

| Cpd No. | Structure | Name | LCMS or ¹H NMR |
|---|---|---|---|
| IA-3 | 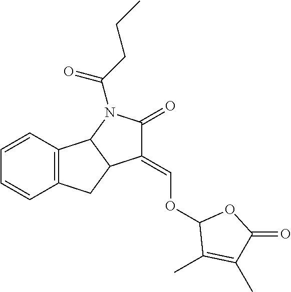 [SMILES: CCCC(=O)N1C(=O)/C(=C/OC2OC(=O)C(C)=C2C)C2Cc3ccccc3C21] | (3E)-1-butanoyl-3-[(3,4- dimethyl-5-oxo-2H- furan-2- yl)oxymethylene]-4,8b- dihydro-3aH-indeno]1,2- b]pyrrol-2-one | Rₜ = 1.11 min (Method A); ES⁺ 382 (M + H⁺) |
| IA-5 | 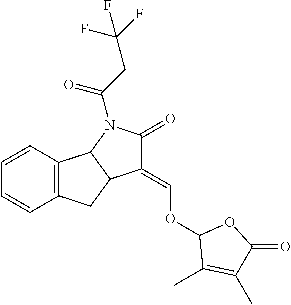 [SMILES: CC1=C(C)C(O/C=C2/C(=O)N(C(=O)CC(F)(F)F)C3c4ccccc4CC23)OC1=O] | (3E)-3-[(3,4-dimethyl-5- oxo-2H-furan-2- yl)oxymethylene]-1- (3,3,3- trifluoropropanoyl)-4,8b- dihydro-3aH-indeno[1,2- b]pyrrol-2-one | ¹H NMR (400 MHz, CDCl₃) (for both diastereoisomers) δ 7.69-7.62 (m, 2H), 7.47 (t, 2H), 7.25-7.18 (m, 4H), 7.01- 6.97 (m, 2H), 6.19 (m, 2H), 5.96 (m, 1H), 5.94 (m, 1H), 3.81-3.73 (m, 2H), 3.43-3.33 (m, 2H), 3.21 (ddd, 2H), 2.07 (m, 6H), 1.58 (m, 6H). |
| IA-6 | 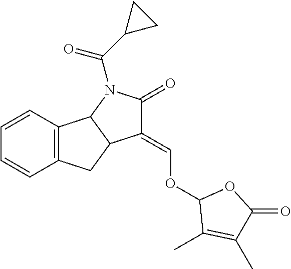 [SMILES: CC1=C(C)C(O/C=C2/C(=O)N(C(=O)C3CC3)C3c4ccccc4CC23)OC1=O] | (3E)-1- (cyclopropanecarbonyl)- 3-[(3,4-dimethyl-5-oxo- 2H-furan-2- yl)oxymethylene]-4,8b- dihydro-3aH-indeno[1,2- b]pyrrol-2-one | Rₜ = 1.07 min (Method A); ES⁺ 380 (M + H⁺) |

### BIOLOGICAL EXAMPLES

Comparative biological studies were conducted on compounds according to the invention (Compounds (IA-1)) and structurally-related compounds known from the prior art: Compounds (P1, P4, P5 and P6) disclosed in WO2012/080115 and (P2 and P3) disclosed in WO2016/193290.

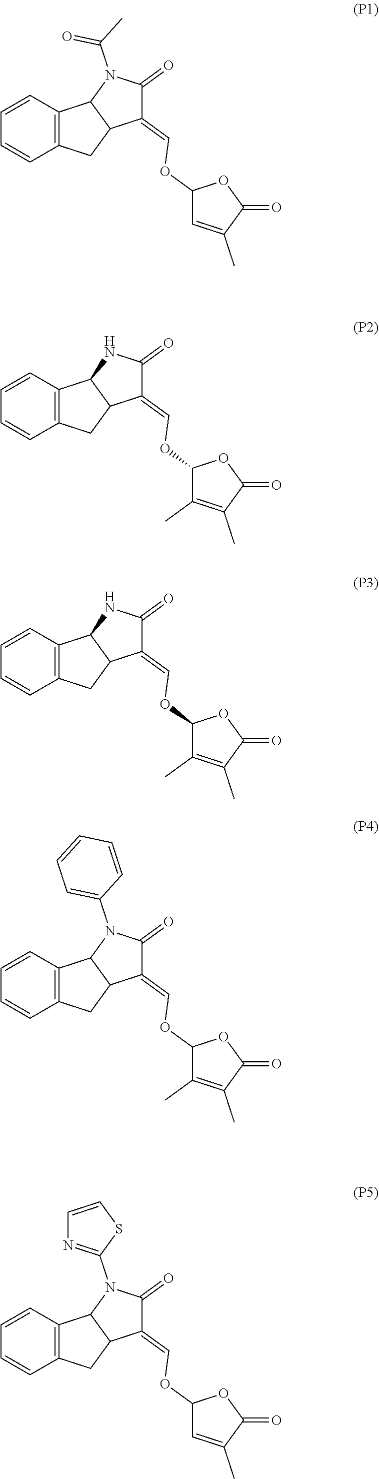

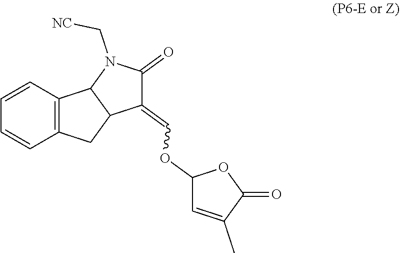 [SMILES: CC1=CC(OC=C2C(=O)N(CC#N)C3c4ccccc4CC23)OC1=O]

### Example B1: Differential Scanning Fluorometry (DSF)

Strigolactone receptor binding studies were undertaken for the compounds of the present invention. Preparation of the maize strigolactone D14 receptor was conducted by cloning gene ID Zm00001d048146 into the pET SUMO expression vector and transforming into BL21(DE3) One ShotR E. coli cells. The transformed cells were cultured to express the D14 receptor protein, which was then purified via his tag purification.

For the DSF assay, 2 μg of purified D14 receptor protein was used in a reaction volume of 25 μl together with 25× Sypro Orange dye, 5× concentrated phosphate buffer and ddH₂O per well of a 96 well plate. The compounds of the present invention were dissolved in DMSO and tested at a final concentration of 5% DMSO.

Thermal shift is a measure of the difference in temperature (ΔT) required to denature a protein with and without a ligand; this provides an indication of the stabilization or destabilization effect caused by the ligand due to ligand-protein binding. To assess the thermal shift, a CFX Connect Real-Time PCR Detection System (Biorad) was used. After an initial 1 min incubation at 20° C. samples were heat denatured using a linear 20° C.-96° C. gradient, at a rate of 0.5° C./30 sec. Compounds were tested in triplicate at a concentration of 200 μM and a protein/DMSO control was included in every plate to calculate the thermal shift. The results in Table 2 are an average of the 3 replicates.

TABLE 2
Thermal shift (ΔT) of compounds (IA-1) and
(P2, P3) on maize strigolactone receptor D14

| | Rate | ΔT |
|---|---|---|
| Cpd No. | (μM) | (% vs control) |
| IA-1 | 200 | 3.8 |
| | 50 | 1.7 |
| | 12.5 | −1.3 |
| IA-7 | 50 | 5.3 |
| | 12.5 | 4.0 |
| IA-8 | 50 | 0.9 |
| | 12.5 | −0.2 |
| IA-10-E | 200 | 16.1 |
| | 50 | 17.1 |
| | 12.5 | 14.1 |
| IA-10-Z | 200 | 19.1 |
| | 50 | 18.1 |
| | 12.5 | 13.1 |
| P2 | 200 | 0.6 |
| | 50 | 0.7 |
| | 12.5 | 0.4 |
| P3 | 200 | 1.7 |
| | 50 | 2.6 |
| | 12.5 | 1.4 |

Compounds of the present invention exhibited a higher ΔT compared to prior art compounds P2 and P3 having no N-substitution. This shows that compounds of the present invention unexpectedly have a superior affinity with the maize strigolactone receptor D14 than close unsubstituted structural analogs.

### Example B2: Dark Induced Senescence of Corn Leaf

It is known that strigolactones regulate (accelerate) leaf senescence, potentially through D14 receptor signaling. Compounds of the present invention (IA) were compared to structurally-related compounds (P) in a corn leaf dark induced senescence assay.

Corn plants of variety Multitop were grown in a greenhouse with relative 75% humidity and at 23-25° C. for 6 weeks. 1.4 cm diameter leaf discs were placed into 24-well plates containing a test compounds in a concentration gradient (100 μM-0.0001 μM) at a final concentration of 0.5% DMSO. Each concentration was tested in 12 replicates. Plates were sealed with seal foil. The foil was pierced to provide gas exchange in each well. The plates were placed into the completely dark climatic chamber. Plates were incubated in the chamber with 75% humidity and at 23° C. for 8 days. On days 0, 5, 6, 7 and 8 photographs were taken of each plates, and image analysis conducted with a macro developed using the ImageJ software. The image analysis was used to determine the concentration at which 50% senescence was achieved (IC50), see Table 3. The lower the value, the higher senescence induction potency.

TABLE 3
IC50 of compounds (IA) and (P) for dark
induced senescence of corn leaf

| | IC50 |
|---|---|
| Compounds | (μM) |
| IA-1 | 0.03 |
| P1 | 0.11 |
| P2 | 9.7 |
| P3 | 7.2 |
| IA-8 | 0.17 |
| P4 | 2.47 |
| IA-10-E | 0.155 |
| IA-10-Z | 0.070 |
| P6-E | 3.71 |
| P6-Z | 0.074 |

Compounds of the present invention exhibited lower IC50 values than their corresponding prior art compounds P (IA-1 compared with P1; IA-8 compared with P4; IA-10 compared with P6). This shows that compounds of the present invention unexpectedly lead to a superior leaf senescence promotion activity than close structural analogs. Inducing leaf senescence may improve nutrient (such as nitrogen or sugar) recycling and remobilization in plants at appropriate timing.

1. A compound of Formula (I): 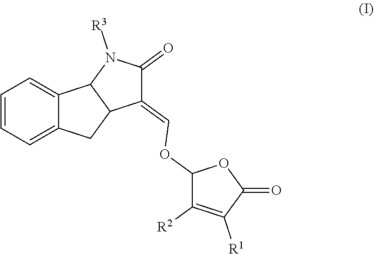
wherein
R¹ and R² are each independently methyl or ethyl; and
R³ is selected from the group consisting of formyl, C₁-C₄ alkylcarbonyl, C₁-C₄ alkoxycarbonyl, C₃-C₈ cycloalkylcarbonyl, C₁-C₄ haloalkylcarbonyl, aryl, heteroaryl, and acetonitrile;
or salts thereof.

2. The compound according to claim 1, wherein R¹ and R² are both methyl.

3. The compound according to claim 1, wherein R³ is selected from the group consisting of C₁-C₄ alkylcarbonyl. C₃-C₈ cycloalkylcarbonyl, C₁-C₄ haloalkylcarbonyl, phenyl, 2-thiazolyl, and acetonitrile.

4. The compound according to claim 3, wherein R³ is selected from the group consisting of formyl, acetyl, phenyl, 2-thiazolyl, and acetonitrile.

5. The compound according to claim 4, wherein R³ is acetyl.

6. The compound according to claim 4, wherein R³ is acetonitrile.

7. The compound according to claim 1, having the structure of Formula (IA-1):

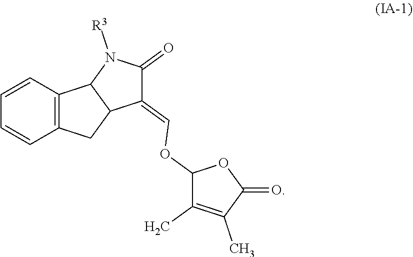

8. A crop yield enhancing composition, abiotic stress management composition, plant growth regulator composition or seed germination promoting composition, comprising a compound according to claim 1, and optionally, an agriculturally acceptable formulation adjuvant.

9. A composition according to claim 8, comprising a further active ingredient.

10. A method for regulating the growth of plants, enhancing the yield of plants, improving the tolerance of plants to abiotic stress factors, accelerating senescence of plant leaves, wherein the method comprises applying to the plant, plant part, plant propagation material, or plant growing locus a compound according to claim 1.

11. A method for promoting the germination of seeds comprising applying to the seeds, or a locus containing seeds, a seed germination promoting amount of a compound according to claim 1.

12. A method for controlling weeds comprising applying to a locus containing weed seeds a seed germination promoting amount of a compound according to claim 1, allowing the seeds to germinate, and then applying to the locus a post-emergence herbicide.

13. Use of the compound of Formula (I) according to claim 1 as a crop/yield enhancer, plant growth regulator or a seed germination promoter.

14. A method of treating a plant propagation material comprising applying to the plant propagation material a compound according to claim 1 in an amount effective to increase the yield, promote germination or regulate plant growth.

15. A plant propagation material treated with a compound of Formula (I) according to claim 1.
